(19) United States
(12) Patent Application Publication
(10) Pub. No.: US 2008/0082148 A1
(43) Pub. Date: Apr. 3, 2008

(54) Laser treatment for skin tightening

(76) Inventor: Eric F. Bernstein, Gladwyne, PA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/898,430

(22) Filed: Sep. 12, 2007

Related U.S. Application Data
(60) Provisional application No. 60/844,108, filed on Sep. 13, 2006.

(51) Int. Cl. A61N 5/06

(52) U.S. Cl. 607/88; 606/9

(57) ABSTRACT

The present disclosure relates to laser treatment of skin imperfections, such as sagging skin. In particular, there is disclosed a method for improving skin laxity of a mammal, by administering to the skin, in a pulsed, scanned, or continuous administration manner, light having a wavelength ranging from 520 to 610 nanometers. The disclosed method may further comprise topically treating the skin prior to and/or after the administration of the light.

This application claims priority to U.S. Provisional Application No. 60/844,108, filed Sep. 13, 2006, which is incorporated herein by reference in its entirety.

The present disclosure is related to the laser treatment of skin imperfections, such as non-ablative remodeling of skin, or tightening to repair sagging skin.

Skin sagging is associated with chronic sun-damage or photoaging. Although sagging skin is also associated with chronological aging, the majority of sagging skin occurs from long-term sun exposure. A murine model of skin aging has shown that ultraviolet A radiation (UVA) causes sagging skin. It has come to bear that this is a significant clinical problem through numerous publications in the lay literature and through web sites. The traditional treatment for sagging skin is face-lift surgery. More recently, radiofrequency treatment has been used to improve sagging skin. Topical agents typically act slowly to improve photodamage but do little to improve sagging skin. In view of the foregoing, there is a need for a treatment that will significantly improve photodamaged skin, including tightening sagging skin.

The present disclosure is directed to a method for treating photodamaged skin by exposing it to electromagnetic radiation, such as laser light within a range of wavelengths. In one embodiment, the method is directed to tighten sagging skin which encompasses irradiating the skin with light having a wavelength sufficient to irradiate blood vessels that are located in the mid-dermis.

The inventor has surprisingly found that light of a particular wavelength is absorbed by blood vessels that likely target hemoglobin in the mid-dermis. While not wishing to bound be by any theory, it is believed that light of sufficient energy stimulates an inflammatory response, which in turns causes the skin and perhaps deeper structures to re-model resulting in skin tightening. The inflammatory response may extend well beyond the depth of penetration of the laser energy.

It has been found that laser light having a wavelength ranging from approximately 520 to 610 nm, such as from 520 to 540 nm and/or from 565 to 610 nm, significantly tightens skin sagging. In one embodiment, wavelengths of 532 nm, 595 nm, or a combination of these wavelengths has been found to be useful for tightening sagging skin. The light can be delivered as a pulse, as a scan or a continuous beam that is swept across the skin giving the effect of a pulse.

The method of the present invention uses laser radiation to stimulate skin tightening by stimulating an inflammatory response directed at blood vessels in the dermis and the associated deeper structures where irradiated. Following laser treatment, a dramatic allergic or urticarial response is present as evidenced by 10 mm hives in the distribution of the laser pulse. These hives may last from one hour up to 4 days depending upon the energy delivered and the relative responsiveness of individual patients. The light is of sufficiently long wavelength to penetrate into the mid-dermis, and the associated inflammatory response is significantly greater in area than the diameter of the treatment beam.

A wavelength of light capable of targeting hemoglobin is selected because blood vessels have numerous associated mast cells that are known for participating in the wound healing response. Once activated as evidenced by the urticarial response to laser treatment, mast cells stimulate migration of inflammatory cells from associated blood vessels. In addition, the vascular endothelial cells of targeted vessels are capable of elaborating numerous cytokines. Sagging skin has been one of the most difficult signs of skin photoaging to improve. Recent advances in laser technology, including the ability to deliver larger amounts of energy deeper into the skin due to larger spot sizes has made skin tightening with visible lasers possible.

There is disclosed a method for improving skin sagging or laxity of skin of a mammal, particularly a human. The method according to the present disclosure comprises a pulsed, scanned, or continuous administration of light in the wavelength range of 520 to 610 nanometers to the skin for a time sufficient to stimulate an inflammatory response in the blood vessels in the dermis of the skin at the point of administration.

It has been shown that beneficial effects occur when the skin is treated with laser light having at least one wavelength of about 532 nm or 595 nm.

Depending on the severity of sagging, it may be desirable to treat various depths of the skin's dermis by using various wavelengths of light on the skin. For example, it has been found that wavelengths ranging (a) from 520 nm to 540 nm or (b) from 565 nm to 610 nm have shown advantageous results. In one embodiment, it is possible to treat the skin with multiple wavelengths, such as wavelengths of light within the ranges provided in (a) and (b).

It is understood that any device capable of producing light within the foregoing wavelength regions may be used. Non-limiting examples of such light producing devices include pulsed dye lasers, solid state lasers, and semiconductor lasers, which are sometimes called diode lasers.

Dye lasers which use complex organic dyes, such as rhodamine 6G, in liquid solution or suspension as the lasing media, are used in one embodiment because they are tunable over a broad range of wavelengths.

In addition, solid state lasers, sometimes referred to as “doped insulator lasers” because their active medium is a solid rod or slab of crystalline insulator that is doped with a small amount of impurity, may be used in the disclosed method. A typical solid state laser that may be used is a Nd³⁺-YAG laser, in which the material is a crystal of yttrium-aluminium-garnate (Y₃Al₅O₁₂), YAG doped with 0.7% by weight of neodymium (Nd³⁺) ions.

This type of solid state laser typically emits its principal laser energies at wavelengths 1.064 μm (infrared). However, the high energies of pulsed Nd³⁺:YAG lasers allow efficient frequency doubling (532 nm), tripling (355 nm), or quadrupling (266 nm). The 532 nm beam is more commonly used in the present disclosure.

According to the present disclosure electromagnetic radiation, such as laser light, within the foregoing wavelength ranges has a spot size from 3-15 mm, such as a 10 mm spot.

In addition, the pulse duration of laser light may range from 0.5 to 500 milliseconds, such as from 1 to 5 milliseconds, and in one embodiment, 1.5 milliseconds.

The average energy or fluence of the electromagnetic radiation, such as laser light used in the disclosed method may range from 3-10 Joules per square centimeter (J/cm²), such as 6-8 J/cm².

In one embodiment, the pulsed, scanned, or continuous administration of longer wavelength of light in (b) may be used in combination with other electromagnetic radiation, such as radiofrequency, fractional laser light, intense pulsed-light using islands of sparing, infrared radiation. Like the administration of light having wavelengths ranging from 565 nm to 610 nm, this other electromagnetic radiation may also be applied in a pulsed, scanned or continuous manner.

In one embodiment, the other electromagnetic radiation may be administered using a low-level source, such as light emitting diodes (LEDs).

In another embodiment, the method according to the present disclosure may comprise topically treating the skin prior to and/or after the administration of the light. In one non-limiting embodiment, topically treating the skin comprises the application of at least one topical agent chosen from alpha-hydroxy acids, retinoids, antioxidants or combinations thereof.

In another embodiment, topically treating the skin also or alternatively comprises at least one mechanical-type process, such as a microderm abrasion or chemical peel.

Also disclosed is a method of improving skin laxity that comprises a pulsed, scanned, or continuous administration of light to the skin for a time sufficient to stimulate an inflammatory response in the blood vessels in the dermis of the skin, wherein the light has a wavelength ranging from 520 to 540 nanometers, from 565 to 610 nanometers or a combination of these ranges.

The present disclosure is further illustrated by the following non-limiting example, which is intended to be purely exemplary of the disclosure.

### EXAMPLE

The following treatments were administered to the skin of the face of ten subjects using a pulsed-dye laser comprising a rhodamine dye. Ten subjects were treated under the following conditions three times at 4-6 week intervals, e.g., 1 treatment every 4-6 weeks for a total of three treatments. Electromagnetic radiation (light) was administered using the following parameters: a wavelength of 595 nm, a 10 mm spot, a pulse duration of 3.0 milliseconds, and an average fluence of 6.0 Joules per square centimeter.

Four weeks following the final treatment, a 38% improvement in skin laxity resulting from skin tightening was noted. Improvement in skin laxity was measured by a reduction in the appearance of fine lines and facial wrinkles determined by physician assessment of the treated areas.

As indicated by these results, a method of treating the skin with laser light according to present disclosure significantly improves laxity of sagging skin.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention.

Other embodiments of the invention will be apparent to those skilled in the art from consideration of the specification. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method for improving skin laxity of a mammal, said method comprising
administering a pulsed, scanned, or continuous light having a wavelength ranging from 520 to 610 nanometers to the skin for a time sufficient to stimulate an inflammatory response in the blood vessels in the dermis of the skin at the site of administration.

2. The method of claim 1, wherein said light has a wavelength of about 532 nm, 595 nm, or both.

3. The method of claim 1, wherein said light has a wavelength ranging from 520 nm to 540 nm or from 565 nm to 610 nm, or both.

4. The method of claim 3, further comprising topically treating the skin prior to and/or after the administration of said light.

5. The method of claim 4, wherein said topically treating the skin comprises the application of at least one topical agent chosen from alpha-hydroxy acids, retinoids, antioxidants or a combination thereof.

6. The method of claim 4, wherein said topically treating the skin comprises at least one process chosen from microderm abrasion and chemical peel.

7. The method of claim 1, wherein said pulsed, scanned, or continuous administration of said light is further combined with other electromagnetic radiation.

8. The method of claim 7, wherein said other electromagnetic radiation is chosen from radiofrequency, fractional laser light, intense pulsed-light using islands of sparing, infrared radiation.

9. The method of claim 8, wherein said other electromagnetic radiation is applied in a pulsed, scanned or continuous manner.

10. The method of claim 9, wherein said other electromagnetic radiation is administered using light emitting diodes (LEDs).

11. The method of claim 9, wherein said electromagnetic radiation is administered using a dye laser, a solid state laser, or a semiconducting laser.

12. The method of claim 11, wherein said electromagnetic radiation has a spot size ranging from 3 to 15 mm.

13. The method of claim 11, wherein said electromagnetic radiation is applied to the skin for a time ranging from 0.5 to 500 milliseconds.

14. The method of claim 13, wherein said electromagnetic radiation is applied to the skin for a time ranging from 1 to 5 milliseconds.

15. The method of claim 11, wherein said electromagnetic radiation has an average fluence ranging from 3-10 J/cm².

16. A method for improving skin laxity of a mammal, said method comprising
administering a pulsed, scanned, or continuous light to the skin for a time sufficient to stimulate an inflammatory response in the blood vessels in the dermis of the skin at the site of administration, wherein said light has a wavelength ranging from 520 to 540 nanometers, from 565 to 610 nanometers or a combination of these ranges.

17. The method of claim 16, further comprising topically treating the skin prior to and/or after the administration of said light.

18. The method of claim 17, wherein said topically treating the skin comprises the application of at least one topical agent chosen from alpha-hydroxy acids, retinoids, antioxidants and combinations thereof.

19. The method of claim 17, wherein said topically treating the skin comprises at least one process chosen from microderm abrasion and chemical peel.

20. The method of claim 16, wherein when said light has a wavelength ranging from 565 to 610 nm, said method further comprises the administration of other electromagnetic radiation.

21. The method of claim 20, wherein said other electromagnetic radiation is chosen from radiofrequency, fractional laser light, intense pulsed-light using islands of sparing, infrared radiation, and is applied in a pulsed, scanned or continuous manner.

22. The method of claim 21, wherein said other electromagnetic radiation is administered using light emitting diodes (LEDs), a dye laser, a solid state laser, a semiconducting laser, or combinations thereof

23. The method of claim 20, wherein said electromagnetic radiation has a spot size ranging from 3 to 15 mm.

24. The method of claim 20, wherein said electromagnetic radiation is applied to the skin for a time ranging from 0.5 to 500 milliseconds.

25. The method of claim 20, wherein said electromagnetic radiation has an average fluence ranging from 3-10 J/cm².
